(19) United States
(12) Patent Application Publication
(10) Pub. No.: US 2018/0021021 A1
(43) Pub. Date: Jan. 25, 2018

(54) HEADSET APPARATUS

(71) Applicant: NEURAL ANALYTICS, INC., Los Angeles, CA (US)

(72) Inventors: Jan B. ZWIERSTRA, Los Angeles, CA (US); Trevor W. DUNLOP, Los Angeles, CA (US)

(21) Appl. No.: 15/653,375

(22) Filed: Jul. 18, 2017

Related U.S. Application Data
(60) Provisional application No. 62/364,120, filed on Jul. 19, 2016.

(51) Int. Cl. A61B 8/08; A61B 5/00; A61B 8/00; A61B 8/10; A61N 7/00

(52) U.S. Cl. CPC A61B 8/488; A61B 8/10; A61N 7/00; A61B 8/4444; A61B 5/6814; A61B 2090/502

(57) ABSTRACT

A headset includes an interface configured to contact a head of a user. The interface is configured to contact one or more locations along a temporal fascia region of the head of the user such that a remainder of the headset is spaced from the head of the user.

### CROSS-REFERENCE TO RELATED PATENT APPLICATIONS

This application claims the benefit of and priority to U.S. provisional patent application Ser. No. 62/364,120, filed Jul. 19, 2016, the content of which is incorporated herein by reference in its entirety.

### BACKGROUND

For devices utilizing a headset whose performance is optimized by remaining stable with respect to a user's head (e.g., a Transcranial Doppler device), alignment and movement of the device during operation of the device can be a concern. For example, if the headset connected to the device is disturbed by muscle movements of the headset user (e.g., by muscles located on the head), the device may become misaligned, resulting in false or inadequate readings or performance by the device. As such, it is desirable that a device remain fixed at an optimal position with respect to a user's head during operation, without being disturbed by muscular movements by the user wearing the headset.

### SUMMARY

According to various embodiments, a headset includes an interface configured to contact a head of a user. The interface is configured to contact one or more locations along a temporal fascia region of the head of the user such that a remainder of the headset is spaced from the head of the user.

In some embodiments, the interface includes at least one mounting foot, the mounting foot for contacting the one or more locations along the temporal fascia region of the head of the user.

In some embodiments, the interface includes a plurality of discrete mounting feet, each of the mounting feet for aligning with a respective one of the one or more locations along the temporal fascia region of the head of the user.

In some embodiments, the plurality of mounting feet include at least three mounting feet.

In some embodiments, the interface includes a single continuous padding for aligning with the temporal fascia region of the head of the user.

In some embodiments, the single continuous padding includes a first continuous padding and a second continuous padding surrounding the first continuous padding.

In some embodiments, a density of the first continuous padding is higher than a density of the second continuous padding.

In some embodiments, the first continuous padding is configured to contact the temporal fascia region and the second continuous padding is configured to contact an area surrounding the temporal fascia region.

In some embodiments, the first continuous padding and the second continuous padding are configured to contact and be restricted to the temporal fascia region.

In some embodiments, the interface includes an inflatable bladder.

In some embodiments, the headset further includes an adjustable top strap configured to tighten and loosen the headset against the temporal fascia region of the head of the user.

In some embodiments, the headset further includes a pad attached to the adjustable top strap, the pad configured to contact a galea aponeurotica region of the head of the user.

In some embodiments, the headset further includes an adjustable rear strap configured to tighten and loosen the headset against the temporal fascia region of the head of the user.

In some embodiments, the headset further includes a probe mount connected to the interface, and a probe connected to the probe mount, the probe mount supporting a probe mounted on the probe mount such that the probe is configured to pan and tilt along the head of the user.

In some embodiments, the probe is configured to be manually panned and tilted along the head of the user.

In some embodiments, the headset further includes a motor assembly connected between the probe mount and the probe for automatically panning and tilting the probe along the head of the user.

In some embodiments, the probe includes a Transcranial Doppler (TCD) probe.

In some embodiments, the headset further includes an ocular device attached to the headset, the ocular device configured to align with and cover one or more eyes of the user.

In some embodiments, the ocular device includes a virtual reality headset.

According to various embodiments, a method of manufacturing a headset comprising providing an interface configured to contact a head of a user, the interface configured to contact one or more locations along a temporal fascia region of the head of the user such that a remainder of the headset is spaced from the head of the user.

### BRIEF DESCRIPTION OF THE FIGURES

Features, aspects, and advantages of the present invention will become apparent from the following description and the accompanying exemplary embodiments shown in the drawings, which are briefly described below.

FIG. 1A is a side view of a diagram illustrating the skeletal anatomy of a human head.

FIG. 1B is a side view of a diagram illustrating the muscular anatomy of a human head.

FIG. 2 is an overhead view of a diagram illustrating a multi-point headset according to various embodiments.

FIG. 3A is a transparent side view of a multi-point headset according to various embodiments.

FIG. 3B is a transparent perspective view of the multi-point headset shown in FIG. 3A according to various embodiments.

FIG. 3C is an opaque perspective view of the multi-point headset shown in FIG. 3A according to various embodiments.

FIG. 3D is a frontal view of the multi-point headset shown in FIG. 3A according to various embodiments.

FIG. 3E is a perspective view of the multi-point headset shown in FIG. 3A including an ocular device according to various embodiments.

FIGS. 4A and 4B are perspective views of a padded headset according to various embodiments.

FIG. 5A is a rear view of a padded headset according to various embodiments.

FIGS. 5B and 5C are frontal perspective views of the padded headset shown in FIG. 5A according to various embodiments.

FIG. 5D is a transparent side view of the padded headset shown in FIG. 5A according to various embodiments.

FIG. 6A is a rear perspective view of an adjustable headset according to various embodiments.

FIG. 6B is a frontal perspective view of the adjustable headset shown in FIG. 6A according to various embodiments.

FIG. 7 is a rear perspective view of an adjustable headset according to various embodiments.

FIG. 8A is a frontal perspective view of an adjustable headset according to various embodiments.

FIG. 8B is a rear perspective view of the adjustable headset shown in FIG. 8A according to various embodiments.

FIG. 9A is a frontal perspective view of a rigid headset according to various embodiments.

FIG. 9B is a rear perspective view of the rigid headset shown in FIG. 9A according to various embodiments.

FIG. 10 is a side view of a mounting foot and attachment mechanism according to various embodiments.

FIG. 11 is a side view of a portion of an inside of a headset according to various embodiments.

FIG. 12 is a perspective view of a headset mounting system according to various embodiments.

FIG. 13 is a bottom view of a mounting foot according to various embodiments.

### DETAILED DESCRIPTION

The detailed description set forth below in connection with the appended drawings is intended as a description of various configurations and is not intended to represent the only configurations in which the concepts described herein may be practiced. The detailed description includes specific details for providing a thorough understanding of various concepts. However, it will be apparent to those skilled in the art that these concepts may be practiced without these specific details. In some instances, well-known structures and components are shown in block diagram form in order to avoid obscuring such concepts.

In the following description of various embodiments, reference is made to the accompanying drawings which form a part hereof and in which are shown, by way of illustration, specific embodiments in which the embodiments may be practiced. It is to be understood that other embodiments may be utilized, and structural changes may be made without departing from the scope of the various embodiments disclosed in the present disclosure.

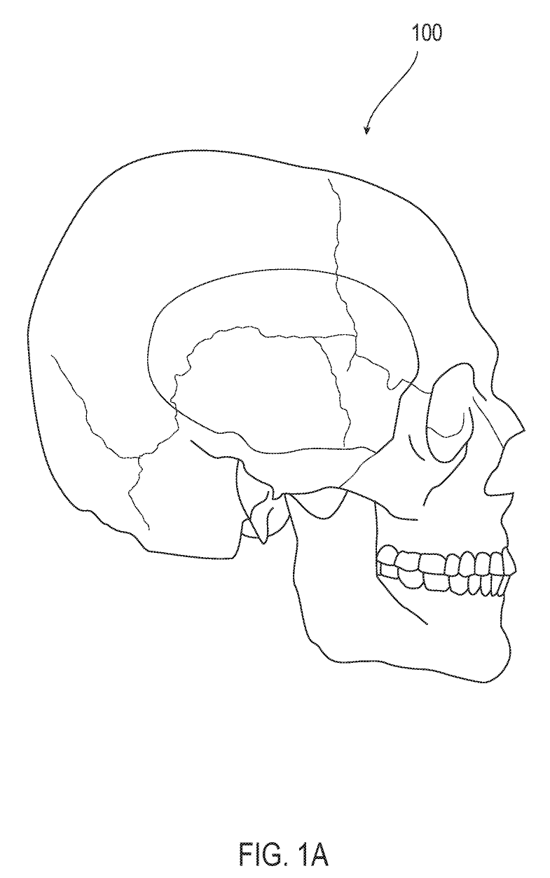

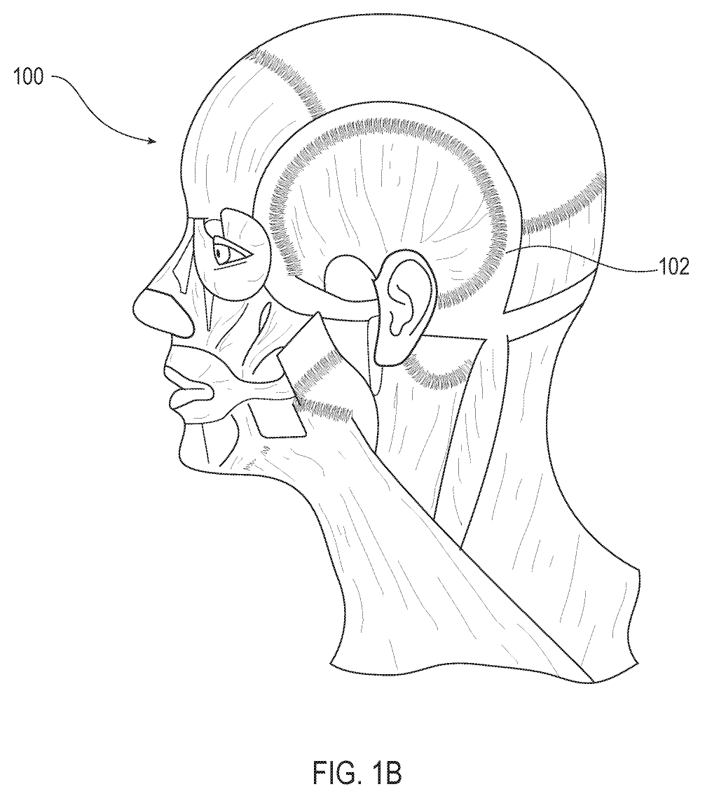

FIG. 1A is a side view of a diagram illustrating the skeletal anatomy of a human head 100. FIG. 1B is a side view diagram illustrating the muscular anatomy of the human head 100.

Referring to FIGS. 1A-1B, the human head 100 includes numerous muscles. However, when a headset including a probe is held fast to a portion of the head 100 that includes muscle tissue, the headset (and therefore the probe) may shift when a user flexes or uses the muscle tissue. As such, affixing the headset to a muscular portion of the head, or strapping the headset around the entire head, may not be desirable when stabilization and alignment of the probe is a priority. As such, in some embodiments, a headset is designed to be affixed to a portion of a human's head that contains minimal or no muscle so that a probe connected to the headset is not disturbed during operation.

For example, the head 100 includes a muscular section referred to as the temporalis, which is a fan-shaped muscle that runs from the side of the skull to the back of the lower jaw and is involved in closing the mouth and chewing. Around the temporalis muscular area, the head 100 includes a bordering ring section referred to as the temporal fascia 102 (shown in FIG. 1B as surrounding the temporalis region). The temporal fascia 102 is an area that has minimal or no muscle, and may include the user's skin and bone only. The temporal fascia region 102 includes only bone and skin and begins at the zygomatic process of the frontal bone, continues along the superior and inferior temporal lines on the parietal bone, and further continues around the back of the ear on the mastoid portion (above the mastoid process) of the temporal bone.

According to some embodiments, a headset is held fast to the temporal fascia 102 of a user so that muscular activation or flexing by the user will not disturb the positioning of a probe attached to the headset. Examples of probes suitable for attaching to the disclosed headset are provided in U.S. patent application Ser. No. 15/187,397 and U.S. patent application Ser. No. 15/399,735, which disclosure is incorporated herein by reference. The temporal fascia 102 provides a stable location for holding a headset to a user's head, as the temporal fascia 102 includes minimal or no muscle tissue. In addition, in some embodiments, because the area of the temporal fascia 102 is primarily a boney structure (e.g., between the superior and inferior temporal lines shown in FIG. 1A), a headset secured to this area provides increased comfort to a user, as the headset applies pressure to bone rather than soft tissue and muscle.

In addition, in some embodiments, a headset can be held fast to the galea aponeurotica region of the head, which is a tough fibrous band that extends over the cranium. The galea aponeurotica also provides a stable location for minimizing movement of the headset and probe during operation. In some embodiments, adjustment of the headset focuses pressure on the galea aponeurotica (e.g., adjustment of the headset downwards into the user's skull). In some embodiments, the headset is held fast to the galea aponeurotica in addition to being held fast to the temporal fascia. In other embodiments, the headset is held fast to the galea aponeurotica instead of to the temporal fascia 102. In yet other embodiments, the headset is held fast to the temporal fascia, but not to the galea aponeurotica. In further embodiments, a rear strap of the headset is included and tracks, without touching, or contacts the external occipital protuberance on the occipital bone.

Accordingly, in various embodiments, a headset is configured to be held fast to a user's head so as to avoid the muscular portions of the head that can shift the headset during operation. For example, in some embodiments, a headset is configured to avoid being held fast to muscles of a user, such as, but not limited to, temporalis (temporal fossa), orbicularis oculi, frontal belly, occipital belly, facial muscles, and neck muscles (e.g., masseter sternocleidomastoid, trapezius, and capitis).

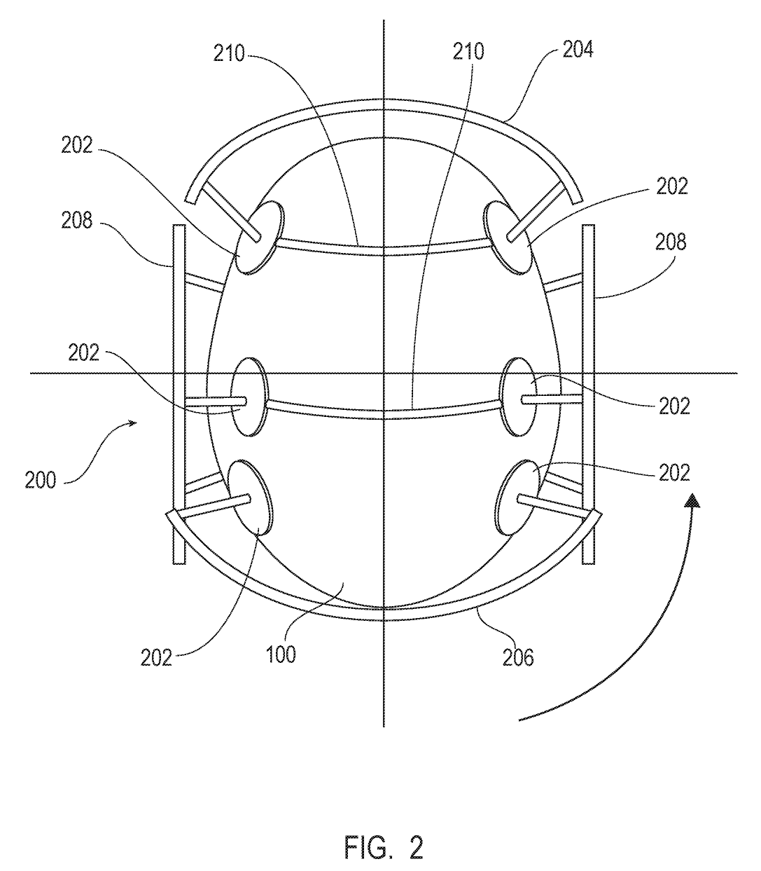

FIG. 2 is an overhead view of a diagram illustrating a multi-point headset 200 according to various embodiments. Referring to FIG. 2, the multi-point headset 200 includes a plurality of mounting feet 202. The multi-point headset 200 further includes a front frame 204 and a rear frame 206. The multi-point headset 200 further includes a plurality of side frames 208, one at each side of the user's head 100. The multi-point headset 200 further includes one or more top straps 210.

In some embodiments, the plurality of mounting feet 202 are pads that are attached to a swivel structure that allows each of the mounting feet 202 to adjust individually to the topography of the user's head 100. In other embodiments, the mounting feet 202 are attached to different types of connectors for allowing adjustability, such as, but not limited to, ball and socket connectors, slide in slots, rotational devices, telescoping mechanisms, or the like. In some embodiments, the mounting feet 202 are generally positioned along the headset 200 such that the mounting feet 202 can be held fast to the areas of a user's head that have minimal or no muscle, as described above. As described herein, the portion of the headsets that are configured to contact the user's head 100 may be referred to as an interface.

In some embodiments, the mounting feet 202 are mounting pads that can be constructed using a variety of techniques or materials. For example, the mounting feet 202 can be open or closed cell foam, self-skinning foams (e.g., latex, urethane, etc.), solid silicone, laminates of multiple materials, large open cell elastomer spring structures, over-molded on rigid support structures, cast-over adjustable supports, constructed with air chambers, rotationally molded, injection molded, co-molded with multiple durometer or density materials, or the like.

In some embodiments, the mounting feet 202 have a shape that is designed to correspond to the contours of the user's head 100. For example, the mounting feet 202 may have a size suitable for securing the headset 200 to the head 100, such as, but not limited to, a range of diameters from about 15 millimeters (mm) to about 60 mm. In other embodiments, the mounting feet 202 may have a size in a range of diameters from about 15 mm to an elongated arc shape that ranges from about 15 mm by about 20 mm to about 15 mm by about 240 mm to about 100 mm by about 240 mm. In further embodiments, each of the mounting feet 202 has a shape suitable for securing the headset 200 to the head 100, such as, but not limited to, a concave shape, a square shape, a circular shape, an irregular shape, or the like. In some embodiments, the mounting feet 202 includes a contacting mechanism that have formable padding for contacting and pressing against the head 100.

In some embodiments, the front frame 204, the rear frame 206, and the side frames 208 are rigid and are part of the overall framework of the multi-point headset 200. In some embodiments, the front frame 204, the rear frame 206, and the side frames 208 are made from any suitable rigid material, such as, but not limited to, hard plastic, metals, aluminum, steel, titanium, magnesium, various alloys, rigid plastics, composites, carbon fiber, fiber glass, expanded foam, compression molded foam, stereolithography (SLA) or Fused Deposition Modeling (FDM)-made materials, Reaction Injection Molding (RIM) molding, acrylonitrile butadiene styrene (ABS), thermoplastic olefin (TPO), nylon, polyvinyl chloride (PVC), fiber reinforced resins, or the like.

In some embodiments, the front frame 204, the rear frame 206, and the side frames 208 include one integrated and continuous frame. In other embodiments, the front frame 204, the rear frame 206, and the side frames 208 are disjointed and connectable pieces. In some embodiments, the headset 200 is made of two rigid pieces (e.g., the headset 200 splits at a midpoint along the front frame 204 and the rear frame 206). In some embodiments, the frame has sufficient rigidity to hold a probe mounted to the frame such that the probe may move about the skull of a person and obtain adequate readings. In other embodiments, the frame holds other medical and non-medical devices that are used and stabilized with respect to a user's head.

In some embodiments, the top straps 210 connect the two side frames 208 and are configured to adjust the headset by tightening the side frames 208 inwards and against the user's head 100 such that the mounting feet 202 are adequately pressed against the user's head 100. As such, in some embodiments, the top straps 210 are made from any suitable non-rigid or soft material for reeling in the side frames 208, such as, but not limited to, elastic fabric, elastic Velcro/hook and loop, neoprene, plastic ratcheting, plastic gear drive, rigid aluminum or plastic raised turnbuckles, elastic silicone with press fit adjustment, wire enclosed within padded straps using a spooling mechanism to tighten, or the like.

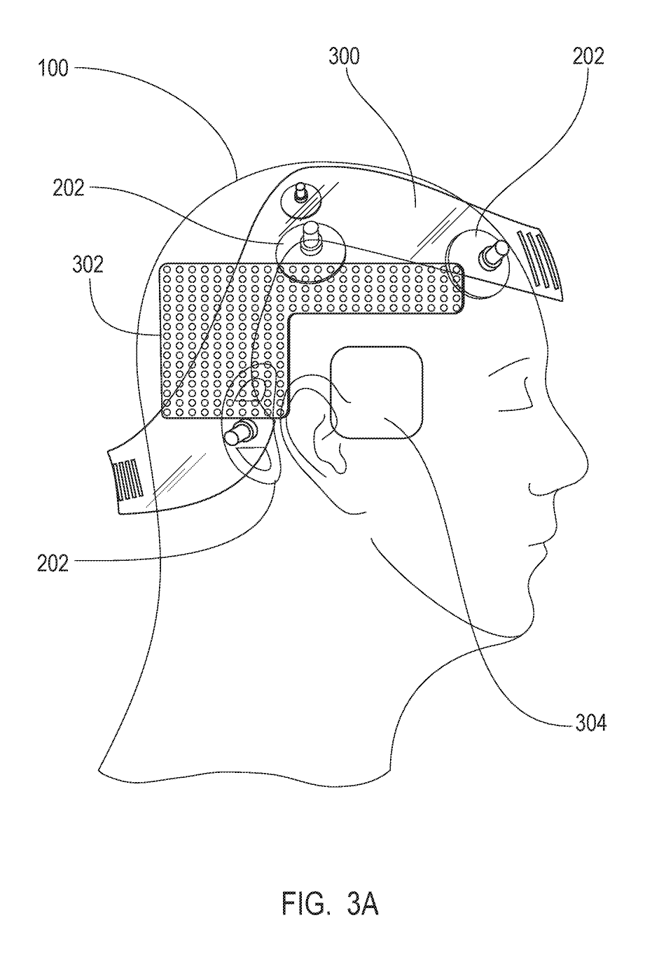

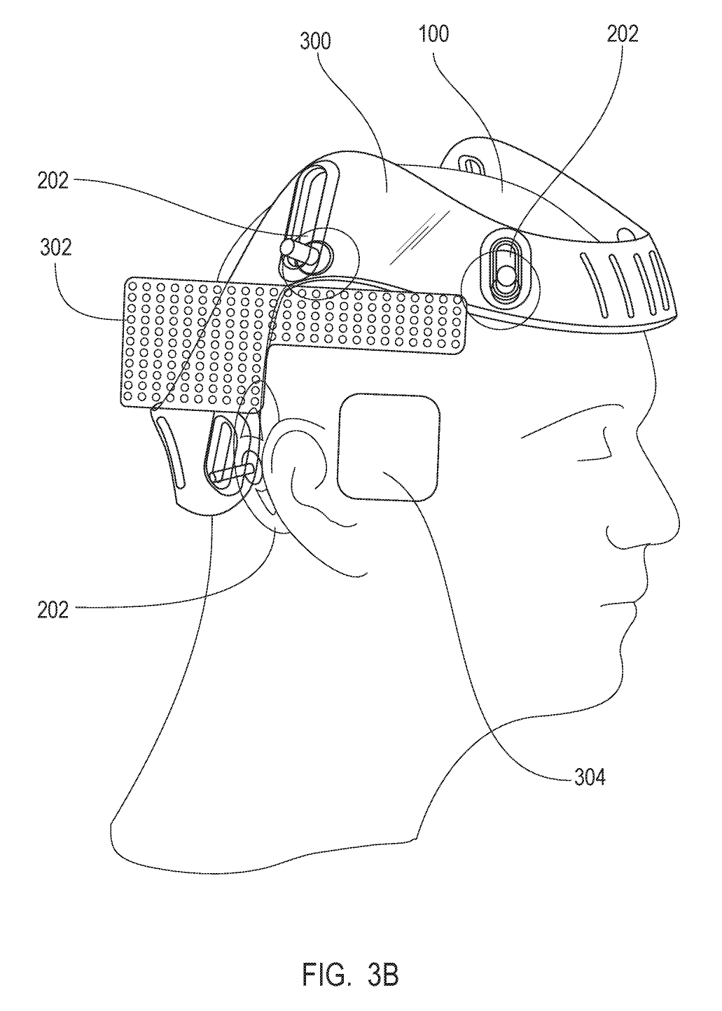

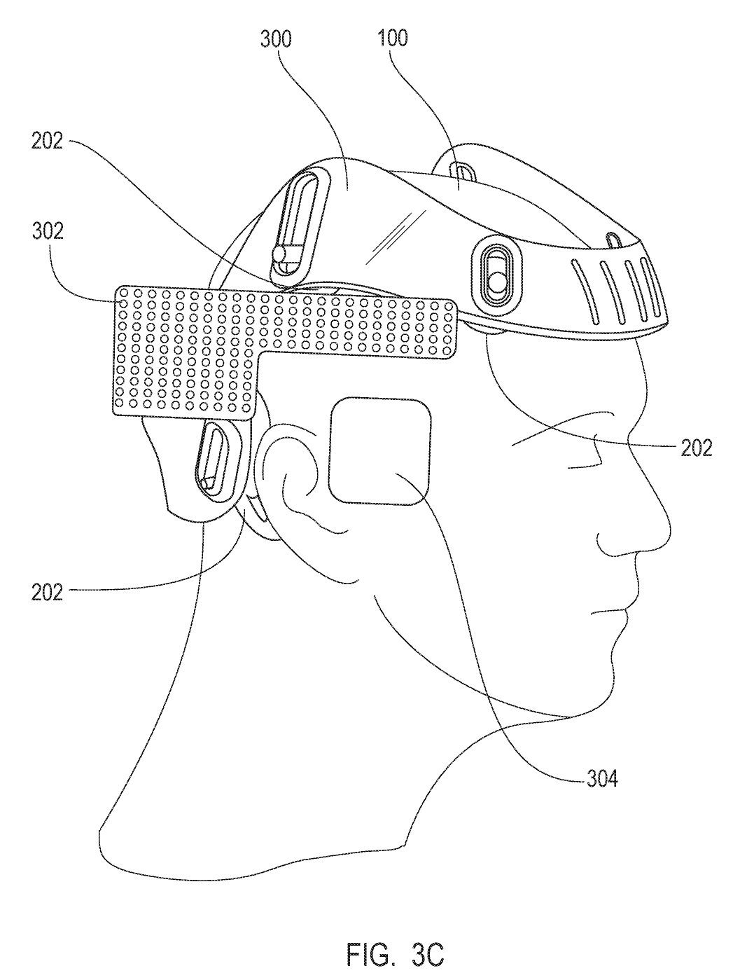

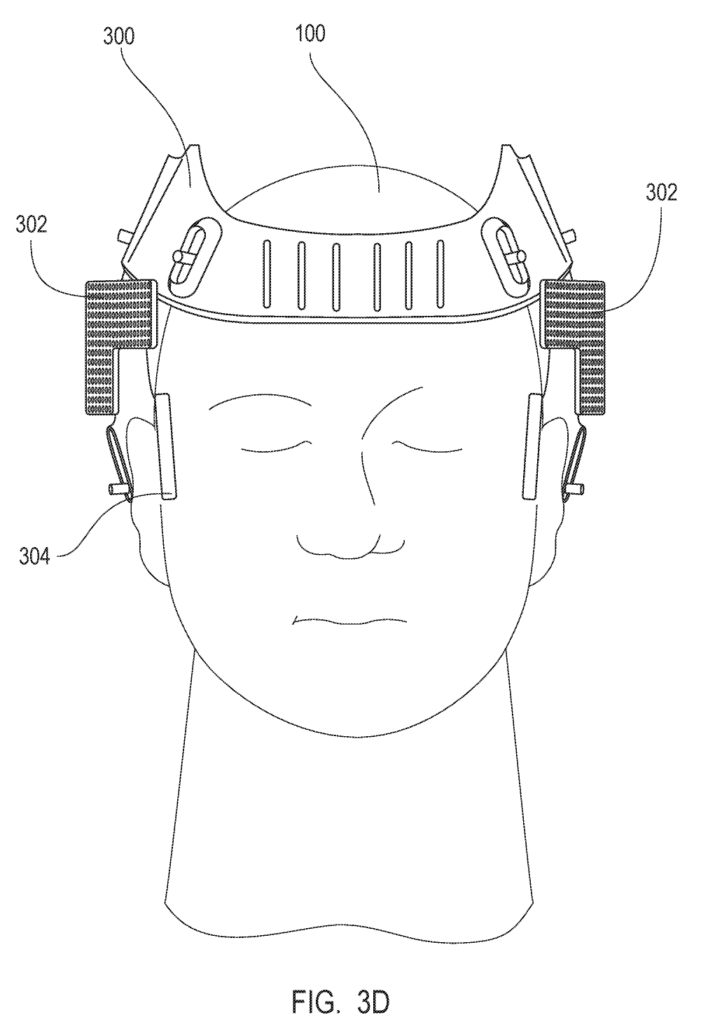

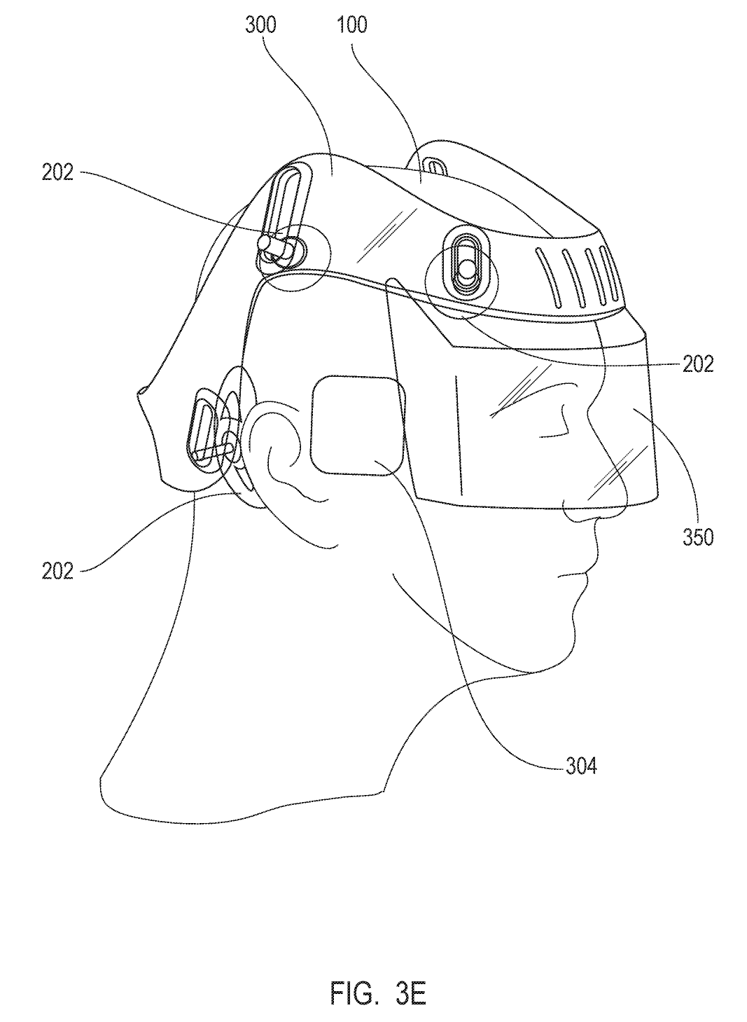

FIG. 3A is a transparent side view of a multi-point headset 300 according to various embodiments. FIG. 3B is a transparent perspective view of the multi-point headset 300 shown in FIG. 3A according to various embodiments. FIG. 3C is an opaque perspective view of the multi-point headset 300 shown in FIG. 3A according to various embodiments. FIG. 3D is a frontal view of the multi-point headset 300 shown in FIG. 3A according to various embodiments.

In some embodiments, the multi-point headset 300 includes the plurality of mounting feet 202. In other embodiments, the headset 300 includes any suitable number of mounting feet 202 for securely affixing the headset 300 to the user's head 100, such as, but not limited to, two mounting feet 202, three mounting feet 202, four mounting feet 202, or more. In some embodiments, the mounting feet 202 are located at one side of the headset 300 (e.g., the left or the right side). In other embodiments, the mounting feet 202 are located at both sides of the headset 300.

In some embodiments, the headset 300 includes three separate mounting feet 202, with each of the mounting feet 202 separated from each other along the frame of the headset 300. In some embodiments, a z-axis distance (e.g., a depth distance along an axis going into the user's head 100) between the forward-most mounting foot (e.g., the mounting foot that is closest to the user's forehead when the headset 300 is worn) and the rear-most mounting foot (e.g., the mounting foot that is closest to the user's ear when the headset 300 is worn) is between about 70 to about 170 mm. In some embodiments, a y-axis distance (e.g., the distance along an axis spanning from the top of the headset 300 to the bottom of the headset 300 between the forward-most mounting foot and the rear-most mounting foot is between about 60 mm to about 160 mm. As such, in some embodiments, each of the mounting feet 202 can shift about 50 mm along the z-axis and about 50 mm along the y-axis for providing adjustability. Furthermore, in some embodiments, the center mounting foot 202 can travel about 50 mm in both the z-axis and y-axis directions, with the maximum y-axis distance that the center mounting foot 202 can travel being the same as that of the forward-most mounting foot 202. In some embodiments, one or more (e.g., each) of the mounting feet 202 are configured to pivot and/or swivel such that surfaces of the mounting feet 202 can properly interface with the user head 100 (e.g., such that the mounting feet 202 sit along a surface of the user head 100). In some embodiments, the headset 300 also defines a space that allows the ear of the user to be exposed to outside of the headset 300. This space allows for ventilation of the ear and provides protection to the ear from the headset 300.

In some embodiments, the headset 300 is used in conjunction with a medical device for use with respect to the user's head 100 (e.g., an ocular monitoring system, a breathing device, a device for monitoring neurological activity, a surgical device, a device for monitoring radioactive traces, or any other device that is optimized when the device itself is not positionally disturbed with respect to a user's head). In other embodiments, the headset 300 is used in conjunction with a non-medical device for use with respect to the user's head 100 (e.g., a virtual reality eyepiece).

As an example of a medical device used with respect to the headset 300, in some embodiments, the multi-point headset 300 includes a continuous frame to which the plurality of mounting feet 202 are attached. The multi-point headset 300 further includes a probe mount 302 attached to the frame of the headset 300. In some embodiments, the probe mount 302 is made of an ultra-rigid material that is not affected by flexing of the headset frame. For example, the probe mount 302 can be made from any suitable rigid material, such as, but not limited to, metal, hard plastic, metals, aluminum, steel, titanium, magnesium, various alloys, rigid plastics, composites, carbon fiber, fiber glass, expanded foam, compression molded foam, SLA or FDM-made materials, RIM molding, ABS, TPO, nylon, PVC, fiber reinforced resins, or the like. In some embodiments, portions of the probe mount 302 are made from flexible, compressible, or elastic materials (e.g., plastics, resins, or the like). In some embodiments, the headset 300 includes the top strap 210 (not shown).

In some embodiments, the probe mount 302 is configured to connect to and control a probe. For example, the probe mount 302 is configured to translate the probe along the surface of the head 100 and to move the probe towards and away from the head 100. In some embodiments, the first end of the probe interfaces with a controller at the probe mount 302, such as, but not limited to, a motor assembly and the like for controlling the probe (e.g., control z-axis pressure, normal alignment, or the like of the probe).

In some embodiments, the probe includes a first end and a second end that is opposite to the first end. In some embodiments, the first end includes a concave surface that is configured to be adjacent to or contact a scanning surface (e.g., the head 100). The concave surface is configured with a particular pitch to focus generated energy towards the scanning surface. In some embodiments, the headset 300 is a Transcranial Doppler (TCD) apparatus such that the first end of the probe is configured to be adjacent to or contact and align along the human head 100 (e.g., a side of the human head), and the first end of the probe is configured to provide ultrasound wave emissions from the first end and directed into the human head 100 (e.g., towards the brain). In other embodiments, the probe is configured to emit other types of waves during operation, such as, but not limited to, infrared waves, x-rays, or the like.

In some embodiments, the second end of the probe is coupled to the probe mount 302. In some embodiments, the second end of the probe includes a threaded section along a portion of the body of the probe, and the second end is configured to be secured in the probe mount 302 via the threads (e.g., by being screwed into the probe mount 302). In other embodiments, the probe is secured in the probe mount 302 by any other suitable connecting means, such as, but not limited to, welding, adhesive, one or more hooks and latches, one or more separate screws, press fittings, or the like.

In other embodiments, the probe is attached to the headset 300 without a motor assembly, such that the probe is configured to be manually operated by an operator while the headset 300 is positioned on the user's head 100. For example, the headset 300 can be affixed to the temporal fascia of the user's head 100, and an operator can manually shift and orient the probe while the probe is activated. Accordingly, the probe attached to the headset 300 will not be affected by muscle movements of the user since the headset 300 is affixed to the temporal fascia region of the user, thereby optimizing performance by the operator of the probe.

In some embodiments, the probe aligns with and contacts a temporal window 304 of the user's head 100. The temporal window 304 includes an area of the user's head 100 at which bone of the skull is thin enough to allow acoustic energy (e.g., ultrasound) to penetrate for allowing monitoring of characteristics of the brain (e.g., cerebral blood flow velocity). Although the temporal window 304 is illustrated as a square, the temporal window 304 can vary in size, shape, and position (e.g., especially from person to person).

Further disclosure regarding probe systems that can be used in conjunction with the headsets described herein can be found in non-provisional patent application Ser. No. 15/399,648, titled ROBOTIC SYSTEMS FOR CONTROL OF AN ULTRASONIC PROBE, and filed on Jan. 5, 2017, which is incorporated herein by reference in its entirety.

FIG. 3E is a perspective view of the multi-point headset shown in FIG. 3A including an ocular device 350 according to various embodiments. Referring to FIG. 3E, in some embodiments, the ocular device 350 is a device that is optimized by maintaining positioning and alignment with a user's eyes (e.g., if the ocular device 350 is shifted with respect to a user's eyes, performance of the ocular device 350 may decline). As an example of a non-medical device use with respect to the headset 200, in some embodiments, the multi-point headset 300 can be used in connection with the ocular device 350. In some embodiments, the ocular device 350 is a virtual reality device configured to provide a virtual experience to the user such that any disturbance of the positioning of the ocular device 350 in front of the user's eyes may cause a degradation in the user's virtual experience.

In other embodiments, the ocular device 350 is a medical device designed to track ocular behavior of the user (e.g., to diagnose whether the user has experienced a neurologic injury or illness such as a concussion). An example of a medical device with respect to the headset, in some embodiments, is a system used to track eye movement and provide diagnostic information on neurological conditions of the subject. In some embodiments, the medical system also displays information for the user to track visually. In other embodiments, the ocular device 350 is an ocular diagnosis or treatment tool for determining or adjusting vision of the user. As an example, the ocular device 350 is a device for correcting imperfect vision of a user (e.g., laser eye surgery). As another example, in some embodiments, the ocular device 350 is an ocular diagnostic tool for determining a vision prescription of a user, presence of one or more eye conditions (e.g., glaucoma, cataracts, ocular hypertension, uveitis, or the like), and so on. In some embodiments, the ocular device 350 is designed to cover and interact with both eyes simultaneously or in sequence. In other embodiments, the ocular device 350 is designed to cover and interact with a single eye (e.g., while the other eye remains uncovered). In some embodiments, because the headset 300 is anchored along the temporal fascia of the user's head, the ocular device 350 can remain stable, and movement of the ocular device 350 can be minimized so that performance of the ocular device 350 is optimized. The ocular device 350 can be provided with any of the headsets described herein.

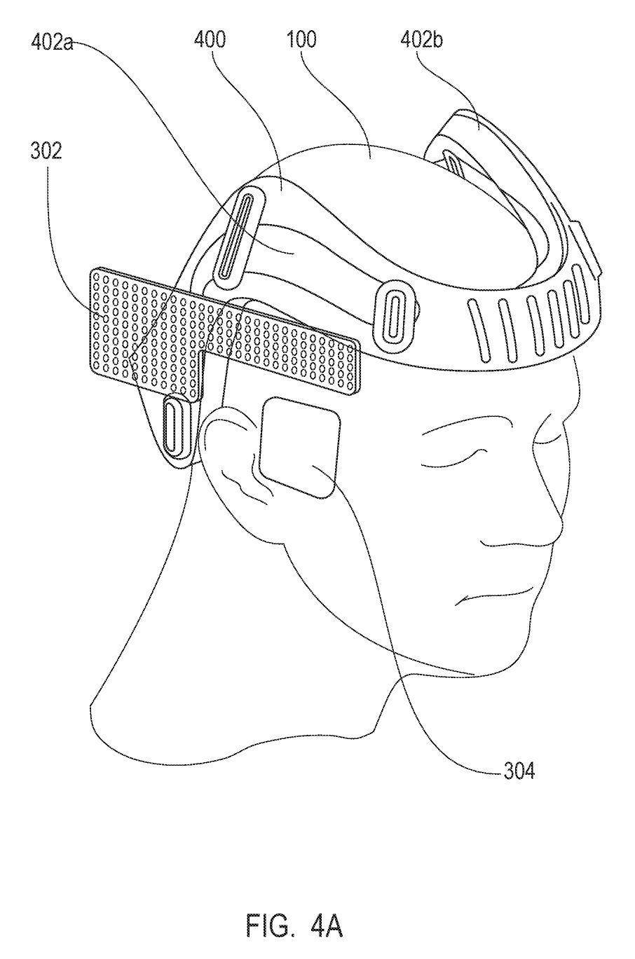

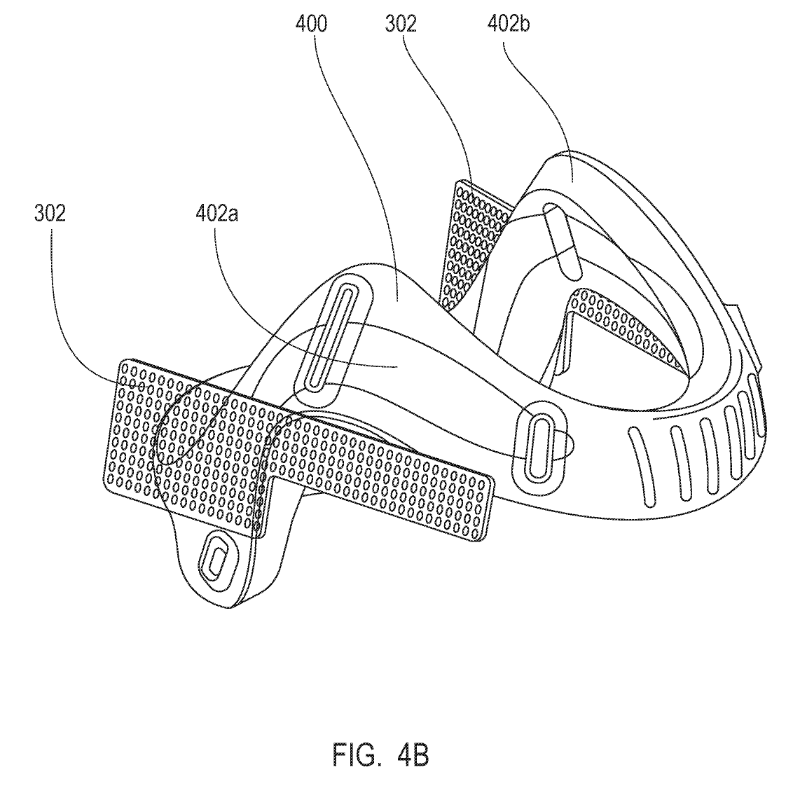

FIGS. 4A and 4B are perspective views of a padded headset 400 according to various embodiments.

Referring to FIGS. 4A and 4B, in some embodiments, the padded headset 400 includes a large continuous padding 402 along the inside of the headset frame. The padding 402 is configured to align with the temporal fascia of the user's head 100. In some embodiments, the padding 402 includes a multi-density padding structure that includes two different paddings 402a and 402b. For example, in some embodiments, the padding 402a has a higher density (e.g., is firmer) than the padding 402b. In some embodiments, the paddings 402a and 402b are continuous along the length of the headset 400. In some embodiments, one of the padding 402a or 402b is continuous along the length of the headset 400, while the other one of the padding 402a or 402b is disjointed at one or more locations along the length of the headset 400 (e.g., has one or more separated sections). In some embodiments, both paddings 402a and 402b are disjointed at one or more locations along the length of the headset 400.

In some embodiments, the multi-density padding allows muscle movement on the head to have negligible impact on the position and stability of the headset 400 when secured to a user's head 100. For example, a base or soft layer of foam flexes easily when not in a completely compressed state, and the contact surface of the soft foam moves along with the muscle, the mid-section of the soft foam moves less, and movement dissipates by the time the foam flexes to the upper section. The higher durometer stiffer foam above provides additional height from the head 100, a platform for soft foam mounting, and compression stability. In some embodiments, the harder foam padding 402a has a firmness in the range of about 1 pound per square inch (psi) to about 60 psi. In some embodiments, the softer foam padding 402b has a firmness in the range of about 0.1 psi to about 10 psi. The psi rating of each of the paddings 402a and 402b is dependent on the shape and structure of the paddings 402a, 402b.

In further embodiments, the paddings 402a, 402b are constructed having compressible or elastic structures (e.g., rubber, urethane, silicone, or the like) that have holes, honeycombs, hollow voids, or the like that effect the firmness of the paddings 402a, 402b. In some embodiments, the higher-density padding 402a aligns with the temporal fascia structure of the head 100. In some embodiments, the padding 402a aligns with the temporal fascia structure of the head 100, while the padding 402b aligns with a region surrounding the temporal fascia structure of the head 100 (e.g., on one side of the temporal fascia structure of the head 100 or on both sides of the temporal fascia structure of the head 100). In other embodiments, both the padding 402a and the padding 402b are aligned with the temporal fascia structure of the head 100 (e.g., the padding 402a and the padding 402b are configured to not extend beyond the temporal fascia structure of the head 100).

In some embodiments, the padding 402 has memory for expanding to fit the contours of the head 100. In some embodiments, the foam of the padding 402 is compressed and expands after the headset 400 is placed on the user's head 100 so that the foam expands to secure the headset 400. In other embodiments, the headset 400 includes an inflatable bladder in place of the padding 402. In some embodiments, the bladder is a hollow void that is filled manually or with a pump. In such embodiments, the inflatable bladder is self-inflating with an internal structure that has a memory and that expands within the bladder to inflate to at least 90% capacity. In further embodiments, inflation is assisted with an integrated pump or an external filling or pumping source. In some embodiments, the inflatable bladder is filled with air, gas, liquid, or any other suitable element for affixing or securing the inflatable padding of the headset 400 to the temporal fascia of the user's head 100.

In some embodiments, the padded headset 400 includes a top strap 210 that extends between the top portion of the headset 400 and over the head 100. In further embodiments, the top strap also includes padding 402 such that the padding aligns with the galea aponeurotica, for further stabilization of the headset 400 on the user's head 100. In further embodiments, the padding along the top strap has the same density as that of the higher-density padding 402a.

In some embodiments, the padding 402 is made from any suitable soft material, such as, but not limited to, closed cell foam, open cell foam, self-skinning open or closed cell foams, cast, aerated, or extruded silicone or urethane, polyurethane gels that are configured to distribute pressure efficiently, or the like.

In some embodiments, the headset 400 including the padding 402 is manufactured by any suitable process for affixing the padding 402 within the headset 400, such as, but not limited to, injection molding, laminating, adhesive mounting (e.g., gluing or bonding), co-molding, co-casting, injection, snapping, by Velcro fastening, by hook and loop fastening, friction fitting, attaching with barbs, using screw bosses, or the like.

In other embodiments, a headset includes a surface forming soft plastic for contacting the temporal fascia with a spring-loaded backing (e.g., a coil, an elastomer, lattice urethane, or the like) to contour to the user's head 100. In yet other embodiments, a headset includes a multi-piece spring-loaded plastic for contacting the temporal fascia, and further includes a living hinge mechanism for flexibility.

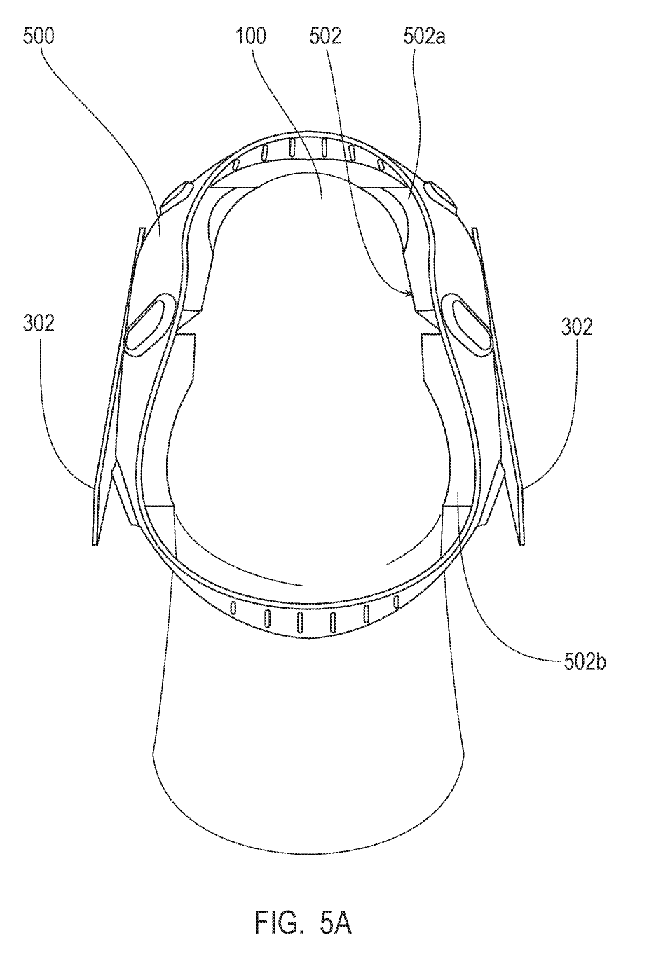

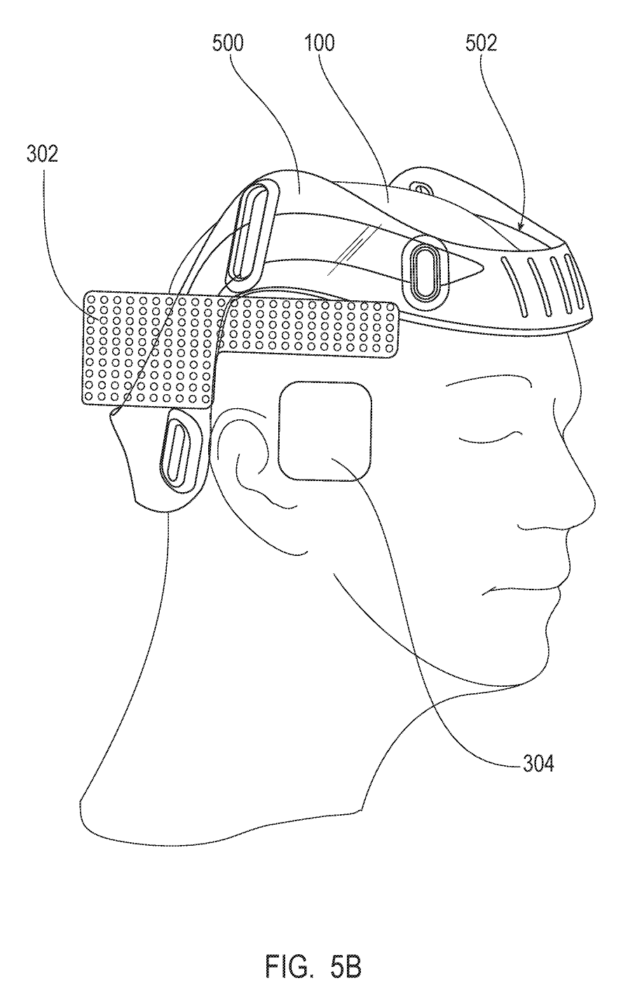

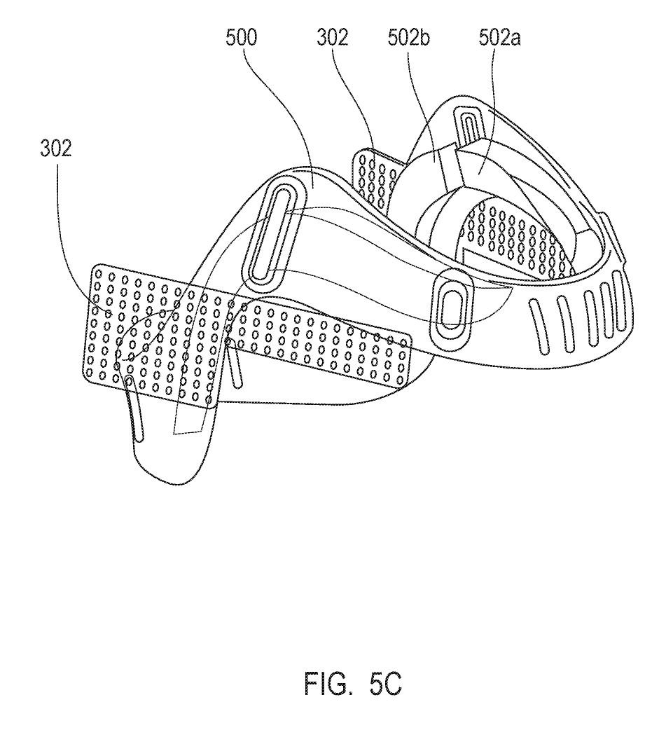

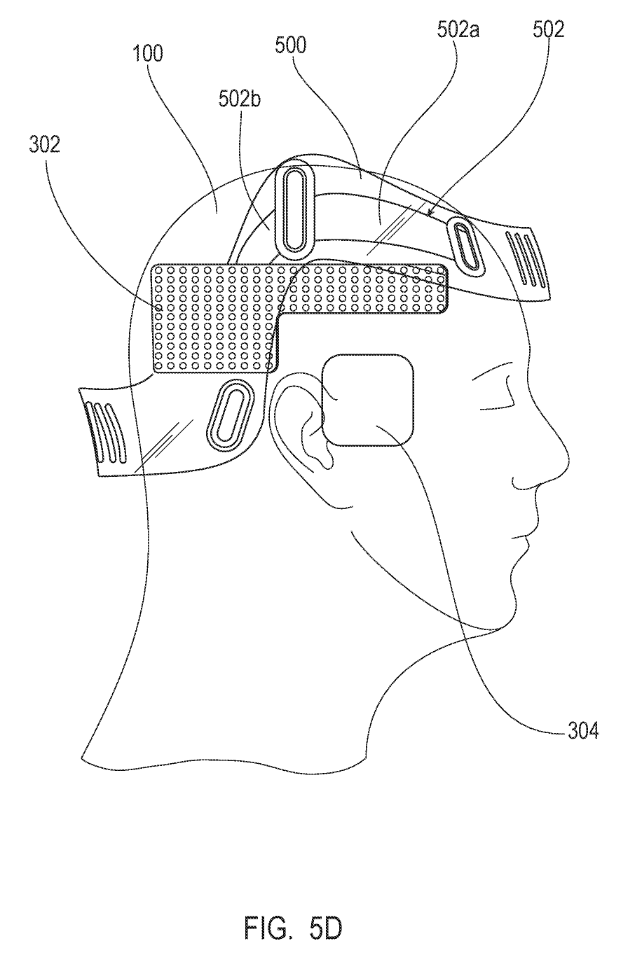

FIG. 5A is a top rear view of a padded headset 500 according to various embodiments. FIGS. 5B and 5C are frontal perspective views of the padded headset 500 shown in FIG. 5A according to various embodiments. FIG. 5D is a side view of the padded headset 500 shown in FIG. 5A according to various embodiments.

Referring to FIGS. 5A-5D, in some embodiments, the padded headset 500 includes a long discontinuous padding 502, such that the padding 502 is separated into two separate sections 502a and 502b that are configured to align with the temporal fascia of the user's head 100. In some embodiments, the sections 502a and 502b have a similar shape, density, size, and so on. In other embodiments, the sections 502a and 502b have different shapes, densities, sizes and so on. In other embodiments, the padded headset 500 includes a long continuous padding (e.g., such that there are not two separate sections 502a and 502b) along the inside of the headset frame. In some embodiments, the padding 502 is discontinuous so as to define more than two sections of the padding 502 (e.g., three or more sections of the padding 502).

In some embodiments, the headset 500 includes the padding 502 designed to align with the temporal fascia of the head 100, but excludes a padding that surrounds the padding 502 (e.g., excludes a padding similar to padding 402b). The padding 502 may be similar to the higher-density padding 402a shown in FIGS. 4A and 4B. In other embodiments, the padding 502 has a higher density than the padding 402a. In some embodiments, the padding 502 has a firmness in a range from about 0.1 psi to about 60 psi.

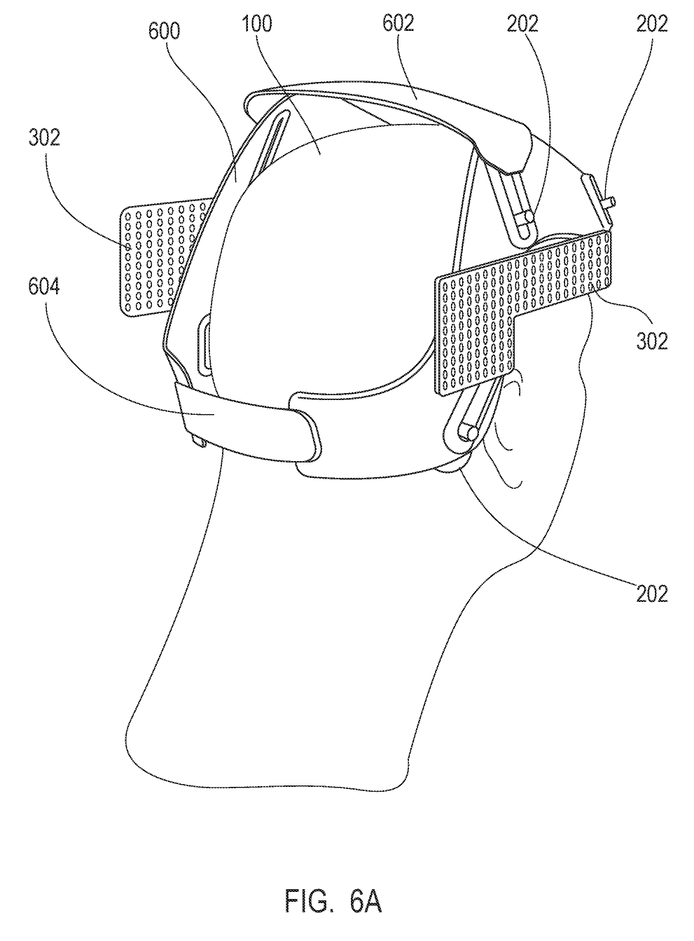

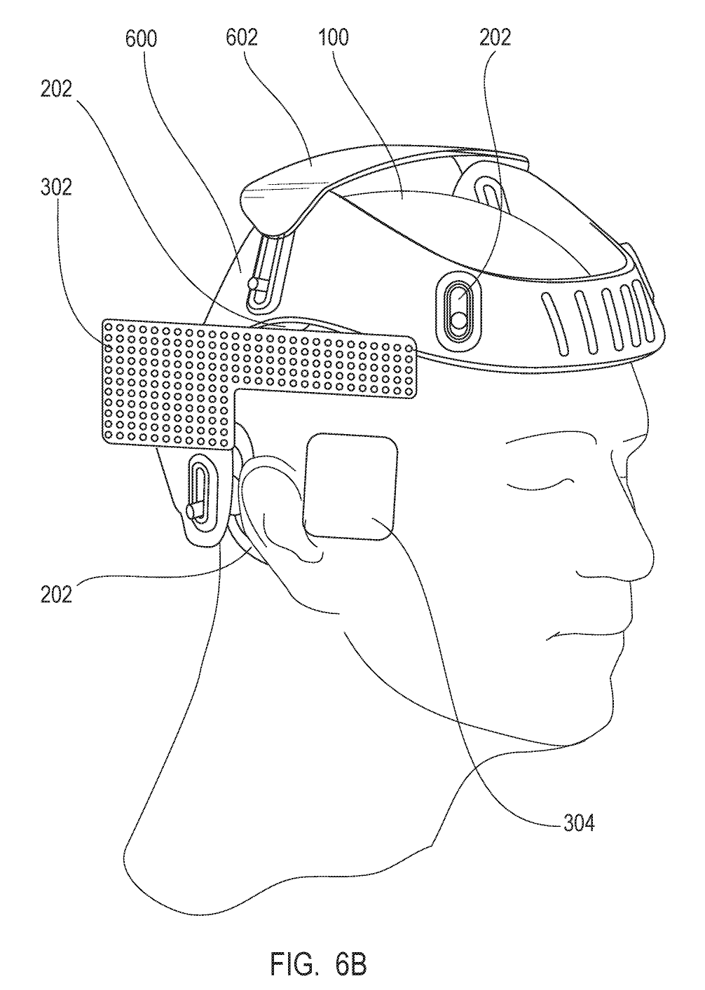

FIGS. 6A is a rear perspective view of an adjustable headset 600 according to various embodiments. FIG. 6B is a frontal perspective view of the adjustable headset 600 shown in FIG. 6A according to various embodiments.

Referring to FIGS. 6A and 6B, the adjustable headset 600 includes a top strap 602 and a rear strap 604 for providing adjustability to the headset 600. In some embodiments, the headset 600 is a multi-point headset. In other embodiments, the headset 600 is a padded headset. In some embodiments, the top strap 602 and the rear strap 604 are adjustable straps that flex the frame of the headset 600 for ensuring a secure fit on the user's head 100. The top strap 602 and the rear strap 604 are made from any suitable soft material for providing adjustability, such as, but not limited to, Velcro, elastic, plastic, or the like. Because the frame of the headset 600 flexes inwards and outwards in adjustment, the headset frame is made from any suitably malleable material that allows for flexing, such as, but not limited to, flexible plastics, polyethylene, urethanes, polypropylene, ABS, nylon, fiber-reinforced silicones, structural foams, or the like.

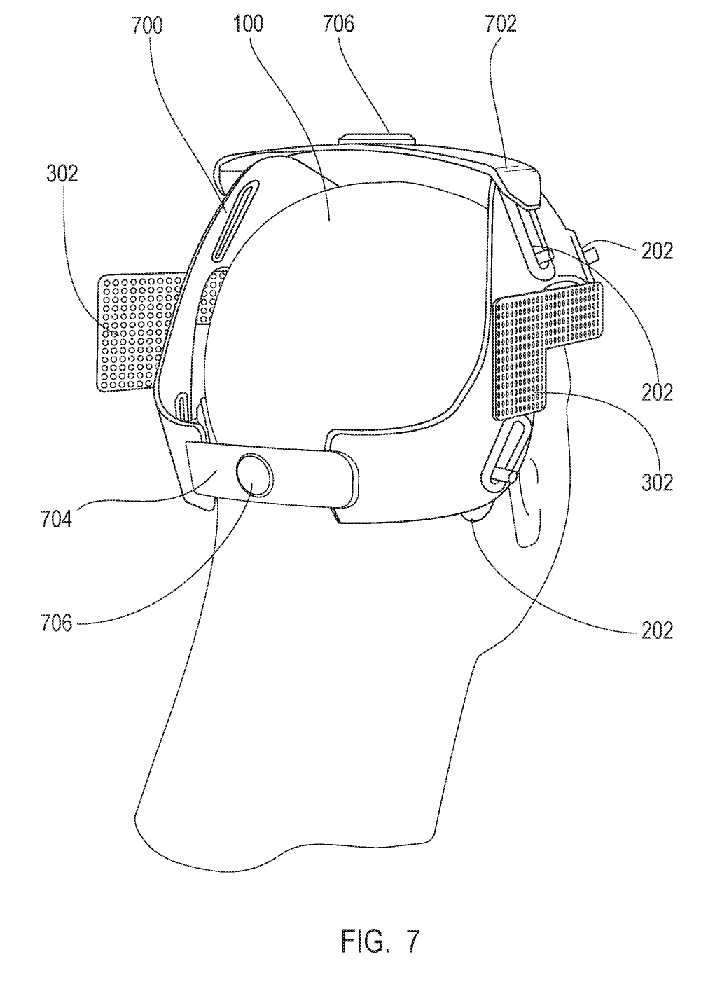

FIG. 7 is a rear perspective view of an adjustable headset 700 according to various embodiments.

Referring to FIG. 7, in some embodiments, the adjustable headset 700 is similar to the adjustable headset 600, except that each of the top strap 702 and the bottom strap 704 includes a knob (e.g., ratchet) 706 for adjusting the respective straps. For example, each of the knobs 706 can be utilized to tighten the headset 700 (e.g., by bending the frame of the headset 700 inwards) or loosen the headset 700 (e.g., by allowing the frame of the headset 700 to bend outwards).

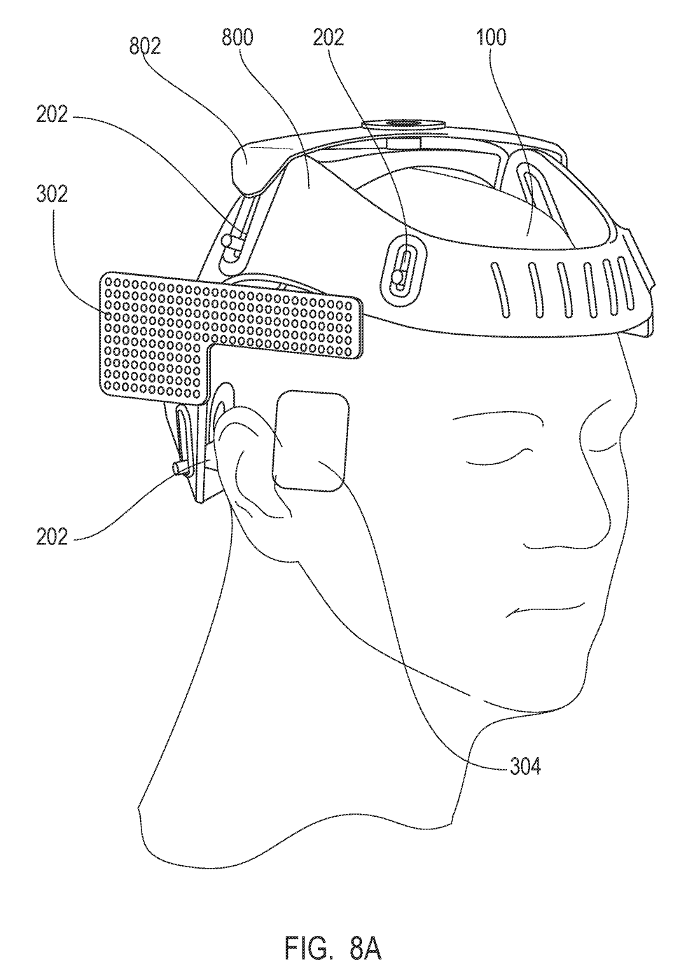

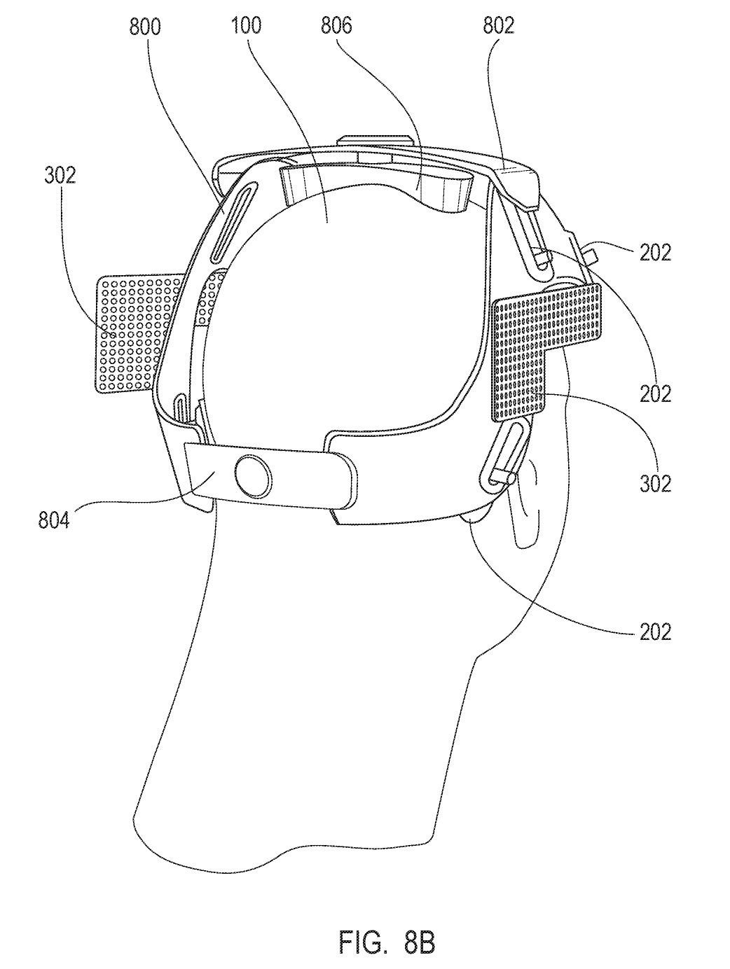

FIG. 8A is a frontal perspective view of an adjustable headset 800 according to various embodiments. FIG. 8B is a rear perspective view of the adjustable headset 800 shown in FIG. 8A according to various embodiments.

Referring to FIGS. 8A and 8B, in some embodiments, the headset 800 includes a top strap 802 and a rear strap 804 and is similar to the headset 700, except that the headset 800 further includes an adjustable pad 806. In some embodiments, the adjustable pad 806 is similar to the padding 402a or the padding 402b. The adjustable pad 806 is configured to apply downward pressure on the user's head 100 (e.g., at the galea aponeurotica) for providing further stability of the headset 800. In some embodiments, the adjustable pad 806 is adjusted via any suitable mechanism for adjusting the downward pressure of the pad 806, such as, but not limited to, a screwing mechanism, a ratchet mechanism, a telescoping mechanism, or the like. In other embodiments, the pad is held fixed in place and is not adjustable.

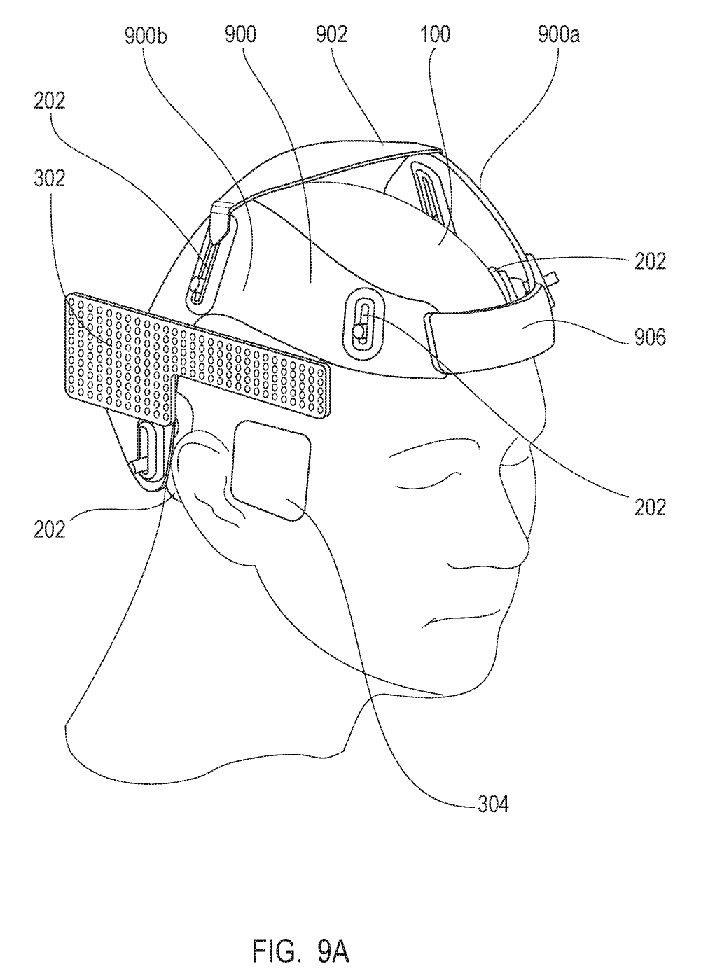

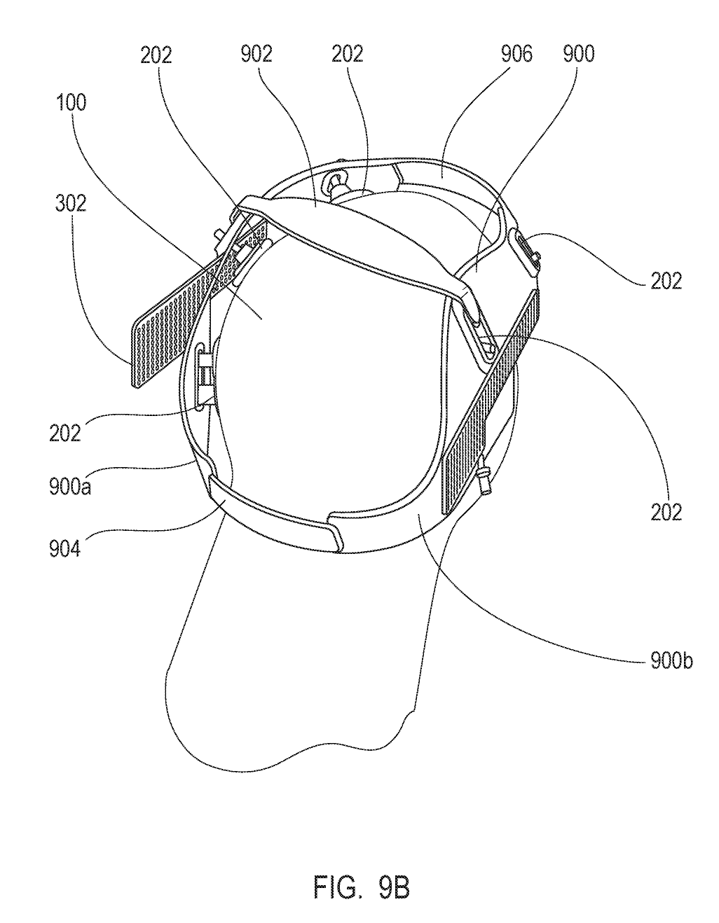

FIG. 9A is a frontal perspective view of a rigid headset 900 according to various embodiments. FIG. 9B is a rear perspective view of the rigid headset 900 shown in FIG. 9A according to various embodiments.

Referring to FIGS. 9A and 9B, in some embodiments, the headset 900 includes a top strap 902, a rear strap 904, and a front strap 906. The headset 900 is made up of a first rigid half frame 900a and a second rigid half frame 900b, with each of the half frames 900a, 900b being separate and distinct components of the headset 900. In some embodiments, the rigid half frames 900a, 900b are made from any suitable semi-rigid material including, but not limited to, hard plastic, metals, aluminum, steel, titanium, magnesium, various alloys, rigid plastics, composites, carbon fiber, fiber glass, expanded foam, compression molded foam, SLA or FDM-made materials, RIM molding, ABS, TPO, nylon, PVC, fiber reinforced resins, or the like.

In some embodiments, the separated half frames 900a and 900b are configured to be placed and secured around the user's head 100. As such, the half frames 900a and 900b are held together and tightened while on the head 100 by one or more of the top strap 902, the rear strap 904, and the front strap 906. As one or more of the straps 902, 904, and 906 are tightened while the headset 900 is placed on the user's head 100, the first rigid half frame 900a and the second rigid half frame 900b flex inwards towards the user's head 100 for securing the headset 900 to the head 100.

In some embodiments, each of the top strap 902, the rear strap 904, and the front strap 906 are made from any suitable non-rigid or soft material for reeling in the rigid half frames 900a and 900b, such as, but not limited to, elastic fabric, elastic Velcro/hook and loop, neoprene, plastic ratcheting, plastic gear drive, rigid aluminum or plastic raised turnbuckles, elastic silicone with press fit adjustment, wire enclosed within padded straps using a spooling mechanism to tighten, or the like. In further embodiments, one or more of the straps 902, 904, and 906 are configured to be adjustable via ratcheting, gears, knobs, or the like. In further embodiments, a mounting structure of a manual or automated probe mechanism (e.g., probe mount 302) is made of an ultra-rigid material so that the probe mounting structure is not effected by any flexing of the rigid half frames 900a, 900b that occur from tightening of the headset 900 on the user's head 100.

In some embodiments, each of the first rigid half frame 900a and the second rigid half frame 900b are designed to include any suitable surface for contacting the user's head 100 when the headset 900 is placed and secured on the head 100. Such surfaces include the plurality of mounting feet 202, the padding 402 or 502, an inflatable bladder, or the like.

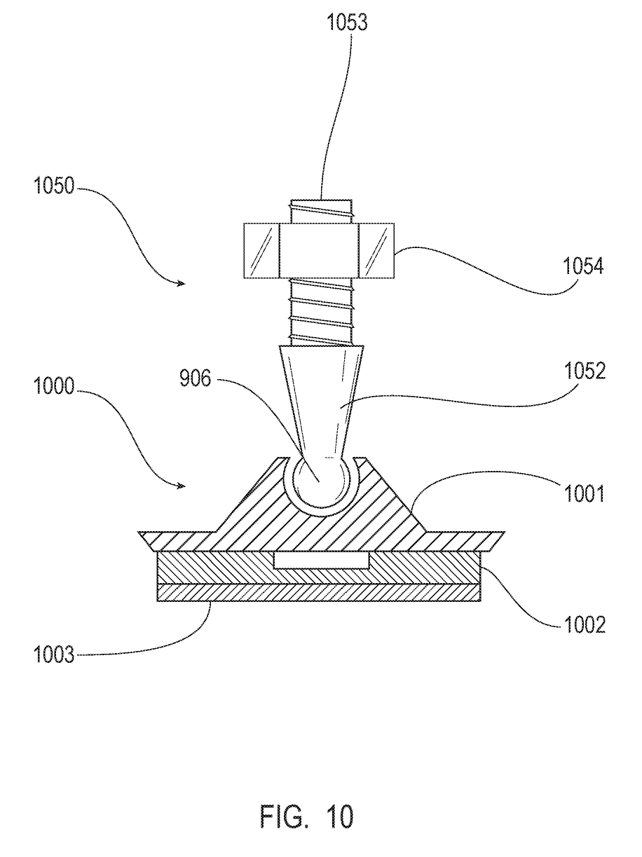

FIG. 10 is a side view of a mounting foot 1000 and attachment mechanism 1050 according to various embodiments. Referring to FIG. 10, the mounting foot 1000 may be similar to the mounting feet 202. In some embodiments, the mounting foot 1000 and attachment mechanism 1050 are incorporated into the headset 300.

In some embodiments, the mounting foot 1000 includes a base 1001, a washer or cover 1002, and a padding 1003. The base 1001 provides a structure for receiving and stabilizing the cover 1002 and the padding 1003. The cover 1002 receives the padding 1003, and the padding 1003 is configured to be placed against the user's head 100. The padding 1003 allows a headset to be comfortably affixed to the user's head 100. In some embodiments, the mounting foot 1000 (e.g., the padding 1003) is pressed against the temporal fascia region of the user's head 100.

In some embodiments, the attachment mechanism 1050 includes a ball and pivot structure 1051, a shaft 1052, a fastener 1053, and a nut 1054. The ball and pivot structure 1051 is attached to the base 1001 to allow free movement of the mounting foot 1000. For example, the ball and pivot structure 1051 can allow rotation (e.g., 360-degree rotation about a center axis of the mounting foot 1000) and lateral pivoting (e.g., about a lateral axis perpendicular to the center axis of the mounting foot 1000). In some embodiments, the shaft 1052 is attached to the ball and pivot structure 1051 so as to allow free range of motion of the ball and pivot structure 1051. The fastener 1053 is connected to the shaft 1052 to allow securing to a headset via tightening of the nut 1054. The fastener 1053 can include a hex screw or bolt. In some embodiments, the shaft 1052 is threaded corresponding to the fastener 1053 to allow a secure connection between the shaft 1052 and the fastener 1053.

In other embodiments, other suitable methods of mounting are utilized, such as a snap, instead of, or in addition to, a screw method. In some embodiments, one or more elements of the mounting foot 1000 and the attachment mechanism 1050 are made from any suitable and lightweight material, such as, but not limited to, plastic or metal, or the like. In some embodiments, ease of moving the joint of the attachment mechanism 1050 depends on the application and little force may be utilized. In addition, the joint may be sufficiently stiff so that it can be pre-set and maintain its position before being placed on the user's head 100.

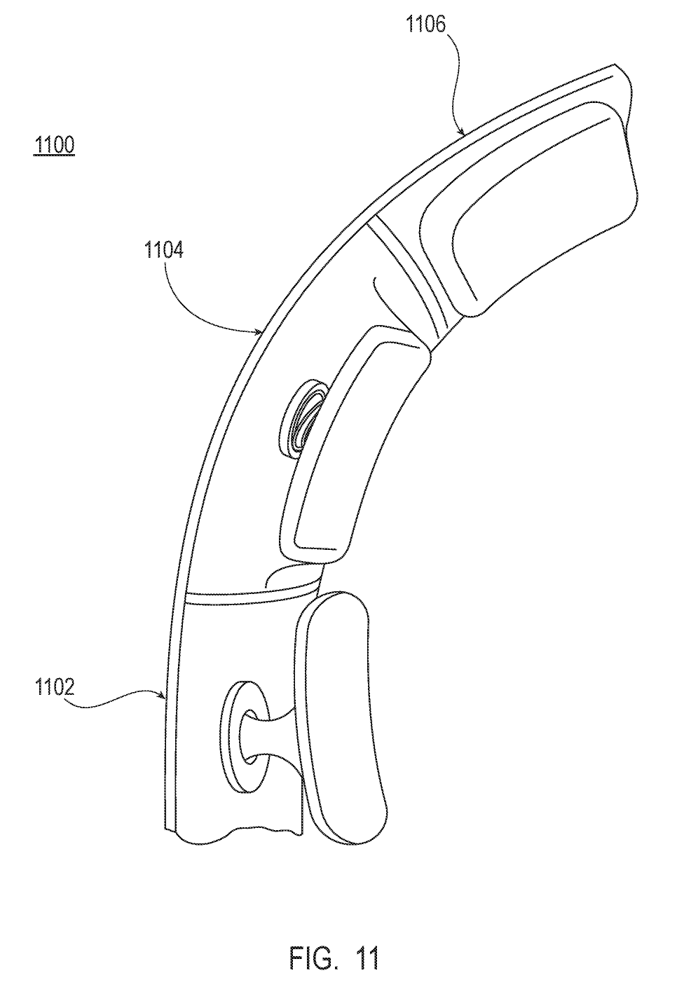

FIG. 11 is a side view of a portion of an inside of a headset 1100 according to various embodiments. Referring to FIG. 11, different foot structures 1102, 1104, 1106 are illustrated. In some embodiments, the different foot structures 1102, 1104, 1106 can be used in connection with any of the headsets described herein. In some embodiments, each of the foot structures 1102, 1104, 1106 is configured to contact the temporal fascia of a user's head. In some embodiments, the different foot structures 1102, 1104, 1106 are implemented together (e.g., as illustrated in FIG. 11) or the different foot structures 1102, 1104, 1106 are implemented alternatively (e.g., implemented as the mounting feet 202). In some embodiments, one or more of the different foot structures 1102, 1104, 1106 are implemented as the mounting feet 202.

In some embodiments, the first foot structure 1102 includes a pivoting structure such that the foot structure 1102 is configured to pivot in one or more directions. In some embodiments, the first foot structure 1102 includes a gel pad for contacting and applying pressure to a user's head (e.g., at the temporal fascia). In some embodiments, the first foot structure 1102 includes a ball joint to facilitate its pivoting function. In some embodiments, the second foot structure 1104 is configured to compress inwards (e.g., in response to pressure applied from a user's head) and extend outwards. The second foot structure 1104 includes a spring to facilitate compression and extension, and the spring constant of the spring is set so as to allow the second foot structure 1104 to maintain sufficient pressure against a user's head to stabilize the headset 1100. In some embodiments, the third foot structure 1106 includes a padded interface having inner contours. In some embodiments, the interface of each of the different foot structures 1102, 1104, 1106 includes a pad. The pads can include any suitable soft material for enhancing user comfortability such as, but not limited to, foam, gel pads, removable padding, dual-density padding, or the like.

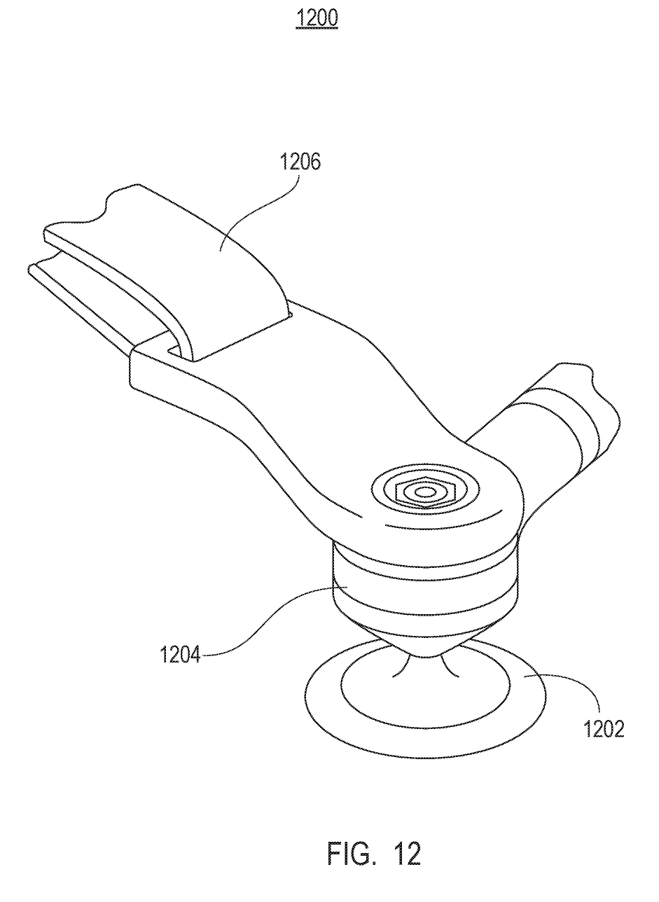

FIG. 12 is a perspective view of a headset mounting system 1200 according to various embodiments. In some embodiments, the headset mounting system 1200 includes a mounting foot 1202, an arm 1204, and a strap 1206. The mounting foot 1202 includes a padding for increasing comfortability of a user. In some embodiments, the mounting foot 1202 is a pivoting structure, as described above, and further has a raised portion (e.g., along an outer rim of the mounting foot 1202). In some embodiments, the arm 1204 is connected to and extends from the mounting foot 1202. The arm 1204 is connected to the mounting foot 1202 such that the arm 1204 is configured to rotate about a center axis of the mounting foot 1202 (e.g., via a ball joint connection). In some embodiments, the arm 1204 is configured to rotate at least 90-degrees about the mounting foot 1202 (e.g., 180 or 360 degrees about the mounting foot 1202). In some embodiments, the arm 1204 has an L-shaped structure extending away from the mounting foot 1202. As such, in some embodiments, the structure of the arm 1204 enables mounting of a headset at a predetermined distance away from a user's head. In addition, the strap 1206 is connected to the arm 1204 for securing the arm 1204 to a headset.

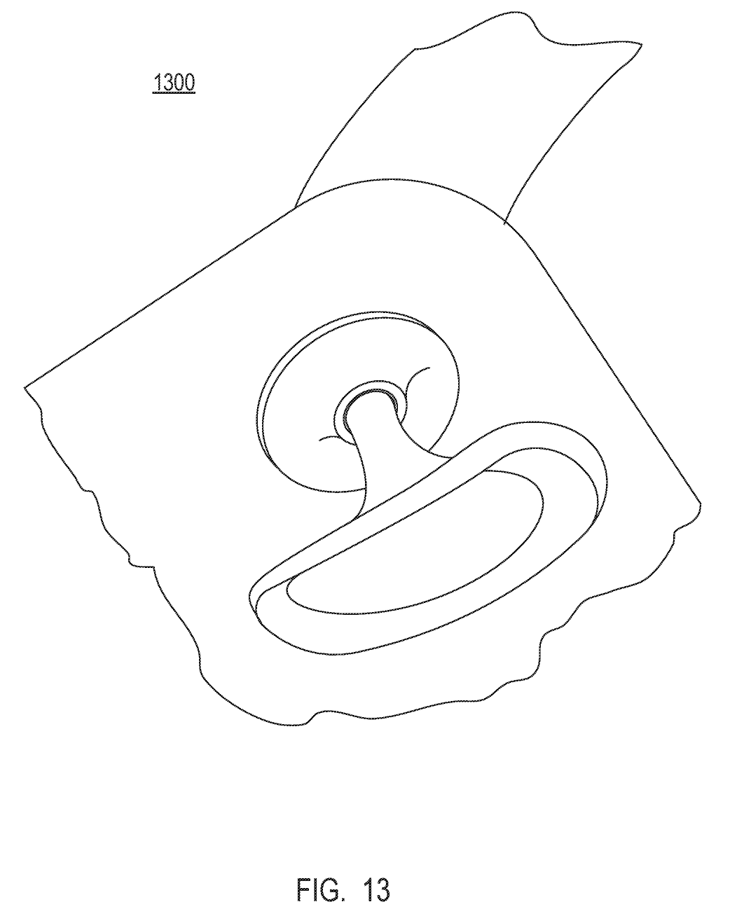

FIG. 13 is a bottom view of a mounting foot 1300 according to various embodiments. In some embodiments, the mounting foot 1300 is a pivoting foot structure including a raised foot and a ball joint, as described above. The ball joint is configured to enable movement of the mounting foot 1300 about at least one axis. In some embodiments, ease of moving the joint depends on the application and it may use little force or be sufficiently stiff so that positioning of the mounting foot 1300 can be pre-set and maintained before being placed on a user's head. In some embodiments, the mounting foot 1300 has a non-circular shape to maximize comfort of a user, such as, but not limited to, oblong, rectangular, oval, or any other suitable shape.

The above used terms, including “held fast,” “mount,” “attached,” “connected,” “secured,” and the like are used interchangeably. In addition, while certain embodiments have been described to include a first element as being “coupled” (or “attached,” “connected,” “fastened,” etc.) to a second element, the first element may be directly coupled to the second element or may be indirectly coupled to the second element via a third element.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout the previous description that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of steps in the processes disclosed is an example of illustrative approaches. Based upon design preferences, it is understood that the specific order or hierarchy of steps in the processes may be rearranged while remaining within the scope of the previous description. The accompanying method claims present elements of the various steps in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description. Thus, the previous description is not intended to be limited to the implementations shown herein but is to be accorded the widest scope consistent with the principles and novel features disclosed herein.

What is claimed is:

1. A headset comprising:
an interface configured to contact a head of a user, the interface configured to contact one or more locations along a temporal fascia region of the head of the user such that a remainder of the headset is spaced from the head of the user.

2. The headset of claim 1, wherein the interface includes at least one mounting foot, the mounting foot for contacting the one or more locations along the temporal fascia region of the head of the user.

3. The headset of claim 1, wherein the interface includes a plurality of mounting feet, each of the mounting feet for aligning with a respective one of the one or more locations along the temporal fascia region of the head of the user.

4. The headset of claim 3, wherein the plurality of mounting feet include at least three mounting feet.

5. The headset of claim 1, wherein the interface includes a single continuous padding for aligning with the temporal fascia region of the head of the user.

6. The headset of claim 5, wherein the single continuous padding includes a first continuous padding and a second continuous padding surrounding the first continuous padding.

7. The headset of claim 6, wherein a density of the first continuous padding is different than a density of the second continuous padding.

8. The headset of claim 6, wherein the first continuous padding is configured to contact the temporal fascia region and the second continuous padding is configured to contact an area surrounding the temporal fascia region.

9. The headset of claim 6, wherein the first continuous padding and the second continuous padding are configured to contact and be restricted to the temporal fascia region.

10. The headset of claim 1, wherein the interface includes an inflatable bladder.

11. The headset of claim 1, further comprising an adjustable top strap configured to tighten and loosen the headset against the temporal fascia region of the head of the user.

12. The headset of claim 11, further comprising a pad attached to the adjustable top strap, the pad configured to contact a galea aponeurotica region of the head of the user.

13. The headset of claim 1, further comprising an adjustable rear strap configured to tighten and loosen the headset against the temporal fascia region of the head of the user.

14. The headset of claim 1, further comprising:
a probe mount connected to the interface; and
a probe mounted on the probe mount such that the probe is configured to move along the head of the user.

15. The headset of claim 14, wherein the probe is configured to be manually positioned along the head of the user.

16. The headset of claim 14, further comprising a motor assembly connected between the probe mount and the probe for automatically positioning the probe along the head of the user.

17. The headset of claim 14, wherein the probe includes a Transcranial Doppler (TCD) probe.

18. The headset of claim 1, further comprising an ocular device attached to the headset, the ocular device configured to align with and cover one or more eyes of the user.

19. The headset of claim 18, wherein the ocular device includes a virtual reality device.

20. A method of manufacturing a headset comprising:
providing an interface configured to contact a head of a user, the interface configured to contact one or more locations along a temporal fascia region of the head of the user such that a remainder of the headset is spaced from the head of the user.
